(19) United States
(12) Patent Application Publication
(10) Pub. No.: US 2004/0101510 A1
(43) Pub. Date: May 27, 2004

(54) Chemokine PARC suppresses specific cytokine production

(75) Inventors: Qin Fu, Baltimore, MD (US); Velizar T. Tchernev, Branford, CT (US); Ebenezer Satyaraj, Hamden, CT (US); Dhavalkumar D. Patel, Chapel Hill, NC (US); Stephen F. Kingsmore, Guilford, CT (US)

Correspondence Address: BANNER & WITCOFF, 1001 G STREET N W, SUITE 1100, WASHINGTON, DC, 20001, US

(73) Assignee: Molecular Staging Inc., New Haven, CT

(21) Appl. No.: 10/660,734

(22) Filed: Sep. 12, 2003

Related U.S. Application Data
(60) Provisional application No. 60/428,944, filed on Nov. 26, 2002.

(51) Int. Cl. A61K 38/19

(52) U.S. Cl. 424/85.1

(57) ABSTRACT

We measured the levels of 78 different cytokines and growth factors in culture supernatants to determine the cytokine profiles of cells treated with PARC. We found that specific cytokines were suppressed by PARC. PARC can be used to treat diseases such as cancer, autoimmune diseases, rheumatoid arthritis, myeloproliferative disorders, and coronary artery disease, which are characterized by too much mitosis.

### FIELD OF THE INVENTION

The invention relates to the field of proliferative cell disorders. In particular, it relates to the cytokines stimulated by chemokine PARC in leukocytes.

### BACKGROUND OF THE PRIOR ART

Ordered arrays of proteins provide an attractive strategy for high-throughput analysis of proteins. To be truly useful for this purpose, however, such arrays must yield sensitive, quantitative, and reproducible measurements of protein levels. It is also desirable that assays on these arrays utilize small sample volumes and be amenable to automated systems for high-throughput processing. There have been a number of recent examples of the use of protein arrays for a variety of applications (1-6). While these approaches have established the feasibility of protein arrays, they have not yet demonstrated practical utility for measuring protein expression levels in a manner analogous to a gene expression array. A microarray consisting of immobilized antibodies is the most straightforward near-term approach for developing a chip for highly parallel analysis of protein levels. Experience with such arrays is limited, and the levels of sensitivity (ca. 10 ng/mL) and multiplexing have been insufficient for quantifying most biological change (7-10).

PARC (pulmonary and activation-regulated chemokine) is also known as AMAC-1 (alternative activated macrophage associated C-C-chemokine), MIP-4 (macrophage inflammatory protein-4), DC-CK1 (dendritic cell-derived chemokine-1) and CCL18. It is expressed mainly by dendritic cells (DC) and macrophages, and is chemotactic mainly for T lymphocytes. PARC is expressed in alveolar macrophages (J Immunol Aug. 1, 1997; 159(3):1140-9), follicular dendritic cells and macrophages (J Leukoc Biol May 2001; 69(5):785-93). PARC has been shown to be chemotactic in vivo for both CD4(+) and CD8(+) T lymphocytes and may therefore be implicated in both humoral and cell-mediated immunity (Genomics Mar. 15, 1999;56(3):296-302).

PARC is mainly induced by Th2-associated cytokines and inhibited by Th1-associated cytokines. PARC is specifically induced in macrophages by cytokines such as, IL-4, IL-13, and IL-10 (mediators of Th2 response). Expression of PARC is inhibited by IFN-gamma (mediator of Th1 response). Peripheral blood monocytes do not express PARC; time course experiments show that monocyte-to-macrophage differentiation is a prerequisite for PARC expression. In vivo, PARC is expressed by alveolar macrophages from healthy persons, smokers, and asthmatic patients. PARC expression is induced by Th2-associated cytokines; thus, PARC may be involved in the APC-dependent T cell development in inflammatory and immune reactions (J Immunol Feb. 1, 1998; 160(3):1411-8). The Th1/Th2 paradigm underlying T-cell polarization has been extended to the corresponding concept of APC polarization. Macrophages as well as DCs can undergo Th1- or Th2-like polarization; APC1 and APC2 thus acquire the capacity to drive the development of naive T cells and the reactivation of resting T cells towards either a Th1 or a Th2 phenotype, respectively. APC1 are classically activated by mediators such as IFN-gamma, TNF-alpha or LPS, while APC2 are alternatively activated by IL-4, IL-10 or PGE. (Skin Pharmacol Appl Skin Physiol September-October 2001; 14(5):272-9. PARC expression is induced by Th2-associated cytokines such as IL-4, IL-13 and IL-10, and is inhibited by Th1-associated cytokines such as IFN-gamma. The Th2-associated expression pattern of PARC in alternatively activated suppressor macrophages in vivo and in vitro and its absence from epidermal Langerhans cells in vivo suggest that it may be involved in inhibition of Th1 reactions and in tolerance induction (Res Immunol September-October 1998; 149(7-8):633-7).

Interestingly, there is a suggestion in the art of a potential application of PARC and its “derivatives” in the treatment of allergy. Allergic reactions are characterized by the infiltration of tissues by activated eosinophils, Th2 lymphocytes, and basophils. The beta-chemokine receptor CCR3, which recognizes the ligands eotaxin, eotaxin-2, MCP-3, MCP-4, and RANTES, plays a central role in this process. PARC, which is known to act as a T cell chemoattractant, exhibits CCR3 antagonistic activity. A modified form of PARC, called Met-chemokine beta 7 (Met-Ckbeta7), is a potent and specific CCR3 antagonist that prevents signaling through this receptor and, at concentrations as low as 1 nM, can block eosinophil chemotaxis induced by the most potent CCR3 ligands. The enhanced activity of Met-Ckbeta7 is due to the alteration of the extreme N-terminal residue from an alanine to a methionine. Met-Ckbeta7 is a more potent CCR3 antagonist than Met- and aminooxypentane (AOP)-RANTES and, unlike these proteins, exhibits no partial agonist activity and is highly specific for CCR3. PARC may therefore use chemokine receptor agonism and antagonism to control leukocyte movement in vivo. Thus, this antagonist and its “derivatives” may be of use in ameliorating leukocyte infiltration associated with allergic inflammation and they have potential therapeutic use in the treatment of allergy (J Immunol Feb. 1, 2000; 164(3):1488-97).

In a study of macrophage- and T-lymphocyte-specific chemoattractants involved in the positioning of immune effector cells during the elicitation phase of contact hypersensitivity (a prototype of a T-lymphocyte-mediated immune reaction), it has been demonstrated that expression of the T-lymphocyte-specific chemokines IP-10 and MIG in epidermis and dermis, and of MDC, PARC and TARC exclusively in the dermis started after 12 hours reaching maximum levels at 72 hours and was associated with infiltration of T cells into the epidermal compartment (Am J Pathol February 2001; 158(2):431-40). PARC expression was significantly increased in lungs affected by hypersensitivity pneumonitis (HP), a lung inflammatory disorder characterized by accumulation of T lymphocytes, in comparison with lungs affected by idiopathic pulmonary fibrosis and controls. Macrophages, DCs, and alveolar epithelial cells were the main sources of PARC. There was a direct correlation between the levels of tissue PARC and the number of lymphocytes in the bronchoalveolar lavage fluids. High levels of PARC were detected in the subacute rather than the chronic phase of HP.

These findings suggest a role for PARC in the pathogenesis of HP (J Leukoc Biol October 2001; 70(4):610-6).

Gene expression of PARC and ELC but not of LARC or SLC has been found in human atherosclerotic plaques, where PARC mRNA was restricted to CD68+ macrophages. The expression patterns of PARC and ELC mRNA in human atherosclerotic lesions suggest a potential role for these two CC chemokines in attracting T lymphocytes into atherosclerotic lesions. (Am J Pathol February 1999; 154(2):365-74).

In livers of patients with chronic hepatitis C, PARC expression has been detected in mononuclear cells mainly in the portal area. The mRNA levels significantly correlated with serum alanine aminotransferase levels (p<0.001). These results suggest that the local production of PARC and RANTES participates in immune responses by attracting naive and active T cells to the portal and periportal areas, respectively (Lab Invest March 2000;80(3):415-22).

CCL18/PARC is selectively induced by staphylococcal enterotoxins in mononuclear cells and PARC levels are enhanced in septic and rheumatoid arthritis. CCL18/PARC protein is spontaneously secreted by PBMC and is selectively induced in PBMC by staphylococcal enterotoxins (SEA, SEB) and IL-4, but not by IFN-gamma and the CXCL8/IL-8 inducers LPS or Concanavalin A. In synovial fluids from septic and rheumatoid arthritis patients, four fold-enhanced CCL18/PARC levels (150 ng/ml) were detected compared to those in crystal-induced arthritis and osteoarthritis. In septic arthritis, the synovial levels of CCL18/PARC were fivefold higher than those of CXCL8/IL-8. Immunochemistry revealed CD68(+) monocytes/macrophages as the main CCL18/PARC-producing cell type in both PBMC and arthritic synovial tissue. In addition, CD1a(+) blood dendritic cells expressed CCL18/PARC. These findings suggest that monocytic cells respond to Gram-positive bacterial infection by the production of CCL18/PARC in the synovial cavity (Eur J Immunol December 2001; 31(12):3755-62 PMID: 11745396).

Using cDNA microarrays, elevated expression of chemokines MIP-1beta and PARC was observed in preterm labour with chorioamnionitis (PTL(+INF)) compared to PTL(−INF) amnion and choriodecidua respectively. Likewise, the cytokines oncostatin-M and PBEF were more highly expressed in PTL(+INF) compared with PTL(−INF) and in spontaneous labour at term compared with Caesarean section at term, respectively. Conversely, inhibin A, TIMP-3 and TIMP-4 were all significantly elevated in PTL(−INF) compared with PTL(+INF). Furthermore, differential expression patterns of classes of genes, grouped according to function (e.g. chemokines), were noted (Mol Hum Reprod April 2002; 8(4):399-408 PMID: 11912289).

In ascitic fluids from patients with ovarian carcinoma, significantly higher levels of CXCL8 and CCL18 (PARC) were detected compared with those in nonovarian carcinoma patients. In contrast to CXCL8, CCL18 was not inducible in carcinoma cell lines. Immunostaining showed CCL18 expression in tumor-infiltrating cells with monocyte/macrophage morphology but not in the ovarian carcinoma cells. Biochemically heterogeneous but biologically active forms of several chemokines are present at different concentrations in ovarian carcinoma ascitic fluid. This points to a delicate balance of chemokines in epithelial ovarian cancer and to a potentially major role for CXCL8 and CCL18 in this tumor (J Biol Chem Jul. 5, 2002;277(27):24584-93 PMID: 11978786).

Cluster analysis of DNA microarray profile of differentially expressed genes in a monkey model of allergic asthma revealed at least five groups of genes with unique expression patterns. One cluster contained genes for several chemokine mediators including eotaxin, PARC, MCP-1 and MCP-3 (Genome Biol 2002;3(5):research0020 PMID: 12049661).

There is a need in the art for improved diagnostic and therapeutic techniques for diseases that are associated with inflammatory processes.

### BRIEF SUMMARY OF THE INVENTION

According to one embodiment of the invention a method is provided for treating a disease characterized by an undesirable amount of cell proliferation. An effective amount of chemokine PARC is administered to a patient who has a proliferative cell disorder, whereby undesirable proliferation of cells is reduced.

According to another embodiment of the invention a method of diagnosing a proliferative cell disorder in a patient is provided. The amount of PARC is determined in a test sample from a patient. The amount determined is compared to an average amount found in a control sample of a population of healthy humans. A decreased amount in the test sample relative to the average amount indicates a proliferative cell disorder in the patient.

According to still another embodiment of the invention a method of stimulating mitosis is provided. An inhibitory molecule for chemokine PARC is administered to a subject in need of augmented mitosis. The inhibitory molecule is selected from the group consisting of antibodies specific for chemokine PARC and antisense molecules which bind to and inhibit transcription of PARC mRNA. Mitosis in the subject is thereby increased.

A still further embodiment of the invention provides a method to monitor the effects of PARC therapy or anti-PARC therapy The amount of one or more proteins in a sample collected from a patient at a first time is determined. The protein is selected from the group consisting of AR, IL-1sR1, IL-2, IL-6sR, LIF, SDF-1a, and fragments thereof. The determination is repeated on a sample collected from the patient at a later time. The amounts measured at the two times is compared. A decreased amount over time denotes an effect of PARC and a increased amount denotes an effect of an anti-PARC inhibitory molecule.

The invention thus provides the art with useful diagnostic and therapeutic methods for diseases characterized by excess mitosis.

### BRIEF DESCRIPTION OF THE DRAWINGS

FIG. 1. Cartoon of immunoassays with RCA signal amplification:

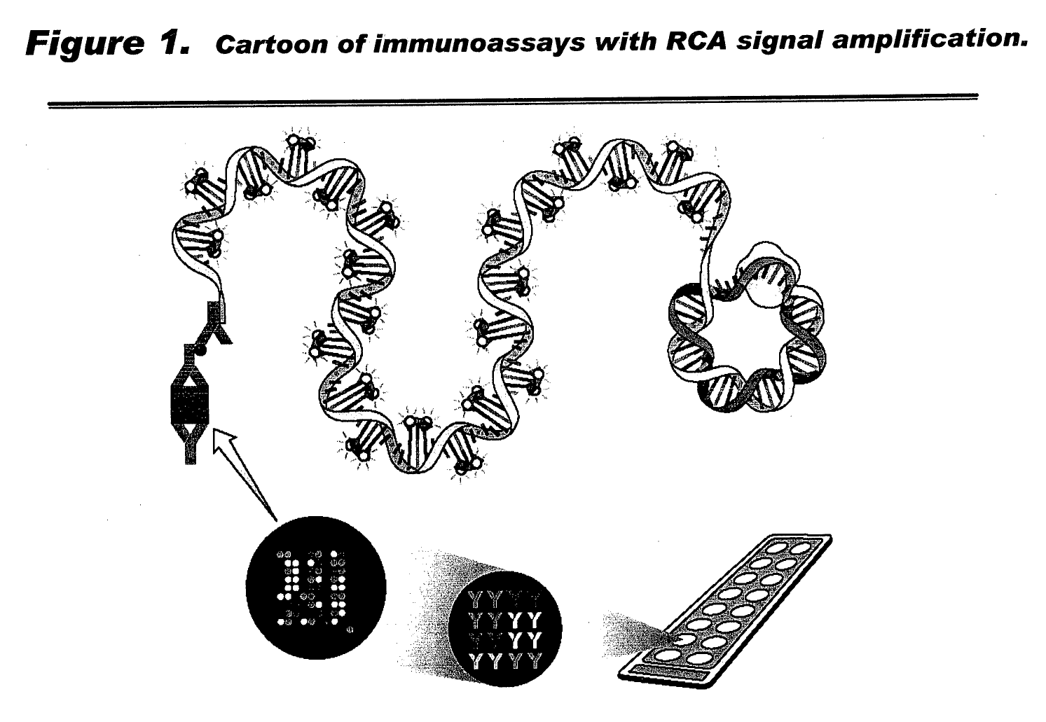

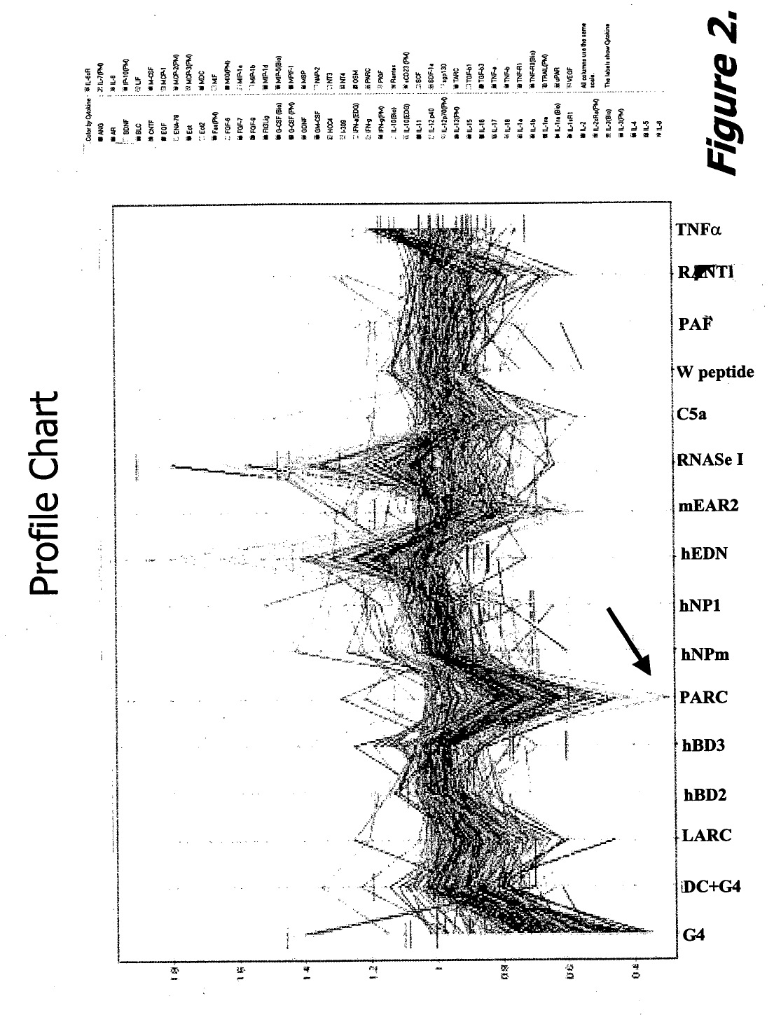

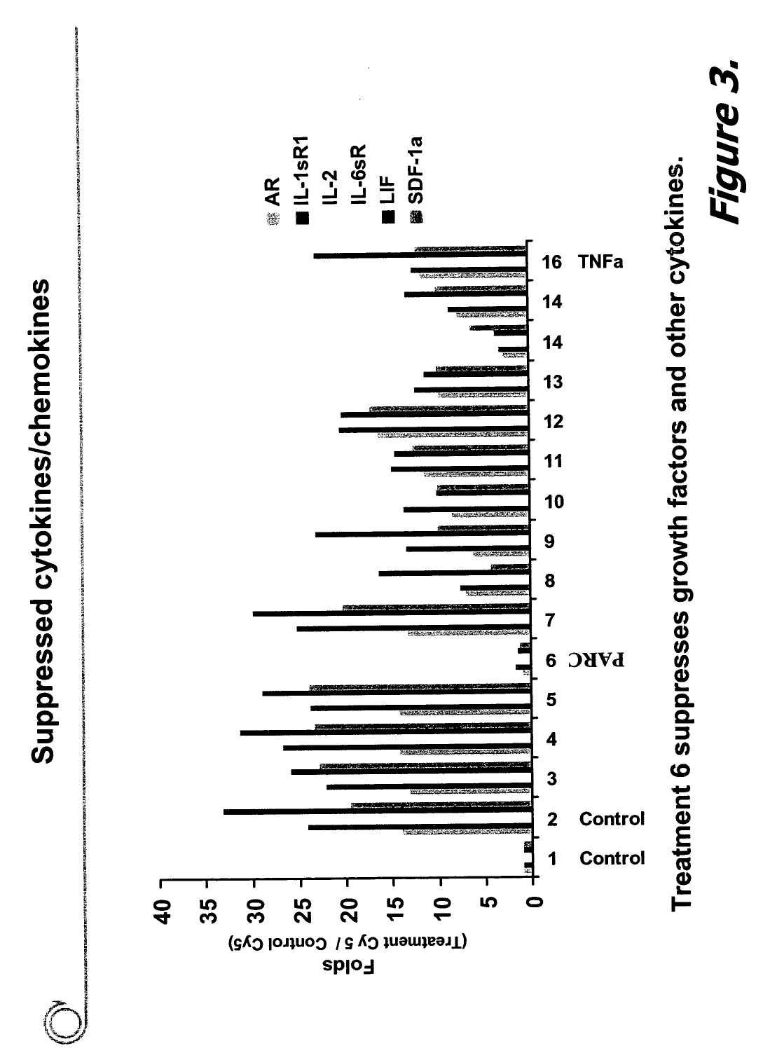

(A). In the adaptation of RCA used for protein signal amplification, the 5′ end of an oligonucleotide primer is attached to an antibody. (B) The antibody-DNA conjugate binds to its specific target molecule; in the multiplexed microarray immunoassay, the targets are biotinylated secondary antibodies and the conjugate is an antibiotin antibody. (C) A circular DNA molecule hybridizes to its complementary primer on the conjugate, and in the presence of DNA polymerase, and nucleotides, rolling circle replication occurs. (D) A long single DNA molecule that represents a concatamer of complements of the circle DNA sequence is generated that remains attached to the antibody. (E) This RCA product is detected by hybridization of multiple fluorescent, complementary oligonucleotide probes. RCA product fluorescence is measured with a conventional microarray scanning device. The amount of fluorescence at each spot is directly proportional to the amount of specific protein in the original sample.

FIG. 2. Cytokine/Chemokines affected by treatment of CD4+ cells.

CD4+ cells were treated for 48 hours with the substance indicated on the x-axis. PARC had a suppressive effect.

FIG. 3. Selected Cytokine/Chemokines affected by PARC.

Conditions were as described in the brief description of FIG. 2.

### DETAILED DESCRIPTION OF THE INVENTION

It is a discovery of the present inventors that chemokine PARC suppresses expression of certain sets of cytokines and chemokines in human leukocytes. Based on the function of these induced cytokines, it is suggested that PARC has novel ani-mitotic activity.

Diseases and conditions that can be advantageously diagnosed and treated according to the present invention include without limitation autoimmune disease, rheumatoid arthritis, cancer, myeloproliferative disease, and coronary artery disease. Other such syndromes are also amenable to the methods of the invention.

Test samples used for performing the diagnostic method are preferably from serum, plasma, blood, lymph fluid, peripheral lymphatic tissue, or blood. Desirably the test sample contains, or has contained, leukocytes, monocytes, dendritic cells, or Langerhans cells. However, it may be desirable that the actual sample upon which the assay is performed be relatively free of cells.

Altered expression of a cytokine can be determined relative to a control sample. The control sample can be obtained from an organ distal to the area of excess mitosis in the test subject. Alternatively, the control sample can be obtained from a subject or subjects not experiencing or evidencing any such disorder. An average value or range can be determined from a population of healthy individuals and used as a control value. Altered expression can be determined at any threshold that is statistically significant. This can be an decrease relative to a control sample of 25%, 50%, or 75%, for example. The threshold can be set to at least on tenth the level of the control sample. Alternatively, the threshold can be set to at least one one-hundredth the level in the control sample.

Altered expression of a cytokine can be determined using either mRNA or protein as an indication of expression level. Preferably the protein will be determined. The determination need not be strictly quantitative. For example, in cases where a cytokine goes from an unexpressed to an expressed state a qualitative assessment may be sufficient. Any assay known in the art for detecting gene expression can be used, either individually or multiplexed. The assays used may involve gene arrays, protein arrays, antibody arrays, Western blotting, ELISA, immunoprecipitation, filter binding assays, hybridization assays, etc. The protein microarray employing a rolling circle amplification for detection described in detail below is preferred, but need not be used. Briefly, capture antibodies are affixed to a solid support in a predetermined pattern (array) and test sample is applied to the array so that proteins (cytokines) in the test sample can bind to antibodies on the array which are specific for that particular protein. Second antibodies are applied which are specific for the same set of proteins as are the capture antibodies. The second set of antibodies can be labeled with a hapten. A third set of antibodies is then applied to the array. The third set of antibodies is specific for the hapten on the second set of antibodies or with the constant region of the second set of antibodies. The third set of antibodies contains an attached oligonucleotide. The oligonucleotide can be used as a primer to amplify a template to create an amplification signal. Preferably the template is a circular DNA such that rolling circle amplification can create a large signal. Alternatively, the second antibody can be directly detectable, for example by rolling circle amplification of an attached oligonucleotide.

If increased mitosis is desired, then a subject can be treated by administering an antibody which specifically binds to a human PARC. The antibody can be a monoclonal or polyclonal antibody. It can be a complete antibody molecule or a fragment. Standard antibody fragments are known in the art and any of these can be used, including Fab, F(ab′)₂. Single chain Fv (ScFv) can also be used. The antibodies can if desired be attached to other moieties, such as therapeutic agents. Single antibodies or cocktails of antibodies can be used. The cocktails can be directed to the same or different cytokines. Antibodies can be administered by any means known in the art, including but not limited to intravenous, intrathecal, directly to the thymus or to a lymph nodes, subcutaneous, oral, and intramuscular. Antisense molecules can also be used which specifically bind to mRNA encoding human PARC and inhibit expression of an PARC.

PARC-treated leukocytes (CD34⁺ cell and monocytes) expressed decreased amounts of a set of cytokine, chemokines, growth factors, and soluble receptors, including AR (amphiregulin), IL-1sR1 (IL-2 soluble receptor 1), I1-2, I1-6sR (IL-6 soluble receptor), LIF (Leukocyte inhibitory factor), SDF-1a (Stromal cell-derived factor-1-alpha), BDNF (brain-derived neurotrophic factor, FGF-6 (fibroblast growth factor-6), Flt-3 ligand (fms-like tyrosine kinase-3 ligand), G-CSF (Granulocyte colony stimulating factor), HCC4 (hemofiltrate CC chemokine 4), IL-12p70.

PARC is a therapeutic target. Inhibitors (in the form of antibodies, small molecular drugs, anti-sense RNA therapy) of PARC can be used to treat diseases associated with too little mitosis. PARC and antibodies and anti-sense molecules thereto can be administered by any technique known in the art. Such methods include, but are not limited to intravenous, intramuscular, subcutaneous, oral, nasal and intrabronchial injections or instillations. Therapeutics based on inhibition of PARC can take the form of proteins, antibody-based therapy or small molecular drugs, anti-sense RNA therapies. The receptors for PARC can also be considered as a therapeutic target for protein therapy, antibody therapy or small molecular drug therapy.

PARC inhibitory molecules can be administered to other individuals in need of an increased mitosis. Such a situation might occur, for example, in the case of wound healing. We utilized highly sensitive antibody based microarray protein chips, which detect 78 cytokines/chemokines simultaneously. Our result demonstrated that a specific set of cytokines/chemokines was suppressed in PARC-treated immature dendritic cells, monocytes and a monocyte cell line.

### EXAMPLES

### Example 1

### Materials and Methods

In Experiment 1, 16 cell culture supernatants (RPMI supplemented with GMCSF and IL-4) were provided by Drs. De Yung and Zack Howard of NCI (NCI Frederick, Frederick, Md. 21702). The cells had been treated as follows:

1. Medium alone without cells, a background control.

2. Medium with cells, a negative control.

3. LARC at 100 ng/ml, human chemokine.

4. hBD2 at 1000 ng/ml, human beta defensin 2.

5. hBD3 at 1000 ng/ml, human beta defensin 3.

6. PARC at 1000 ng/ml, human chemokine.

7. hNPm at 1000 ng/ml, natural human neotrophil defensins (a), mixture of hNP1, hNP2 and hNP3 and isolated from the granules of polymorphonuclear leukocyte.

8. hNP1 at 1000 ng/ml, human neutrophil protein, alpha defensin.

9. hEDN at 1000 ng/ml, human eosinophil derived neurotoxin.

10. mEAR2 at 1000 ng/ml, mouse protein, no effect on human cells and is a negative control.

11. RNase1 at 1000 ng/ml, human RNase 1, eosinophil derived, It can strongly activate iDC and is a control for iDC maturation.

12. C5a at 10 nM, complement factor 5a.

13. W pep. at 100 nM, hexapeptide.

14. PAF at 10 ng/ml, platelet activating factor.

15. RANTES at 100 ng/ml, human chemokine.

16. TNFa at 50 ng/ml, a positive control.

In Experiment 2, 84 cell culture supernatants (RPMI supplemented with GMCSF and IL-4) were provided by Dr. De Yung and Dr. Zack Howard of NCI (NCI Frederick, Frederick, Md. 21702). Samples were divided into following 5 groups:

1. Group 1: (Time-course, 36 samples) monocyte-derived DCs and CD34-derived DCs treated with RNase 1, HEDN (Rnase 2) or RNase 3 for the following times: 0, 2 or 3, 6, 12, 24, and 48 hours.

2. Group 2: (Concentration-dependence, 29 samples) monocyte-derived DCs and CD34-derived DCs treated for 48 hrs with 10, 100, 500, or 1000 ng/ml of RNase 1 or HEDN (Rnase 2); or with 1000 or 3000 ng/ml of RNase3.

3. Group 3: (RNase activity-dependence, 6 samples) CD34-derived DCs treated with 1000 ng/ml RNase 1 or 2 in the presence of ribonuclease inhibitor.

4. Group 4: (Cell type specificity, 8 samples) lymphocytes treated with RNase 1 or hEDN (Rnase 2). Monocyte cell lines treated with RNase 1, hEDN (Rnase 2) or RNase 3.

5. Group 5: (independent RNase source, 5 samples) Monocytes treated with 1000 ng/ml RNase 1, HEDN (Rnase 2) or RNase 3.

Microarray manufacture: Antibody microarrays were printed using a Packard Biosciences (Downers Grove, Ill.) BCA-II piezoelectric microarray dispenser on cyanosilane-coated glass slides divided by Teflon boundaries into sixteen 0.5 cm diameter circular subarrays. Monoclonal antibodies (commercially available) for 78 cytokines were dispensed in quadruplicate at a concentration of 0.5 mg/ml. Printed slides were blocked as described [21] and stored at 4° C. until use. Batches of slides were subjected to a quality control consisting of incubation with a fluorescently-labeled anti-mouse antibody, followed by washing, scanning and quantitation. Typically, the coefficient of variability (CV) of antibody deposition in printing was <5%.

RCA Immunoassay: The assay was performed by a liquid-handling robot (Biomek 2000, Beckman Instruments, Fullerton, Calif., which was enclosed in an 80% humidified, HEPA-filtered, plexiglass chamber. For each sample, duplicates were tested either neat or diluted 1:10. 20 μl of samples was applied to each sub-array and immunoassays with RCA signal amplification were performed as described [21] Slides were scanned (GenePix, Axon Instruments Inc., Foster City, Calif.) at 10-μm resolution with laser setting of 100 and PMT setting of 550. Mean pixel fluorescence were quantified using the fixed circle method in GenePix Pro 3.0 (Axon Instruments, Foster City, Calif.). The fluorescence intensity of 8 microarray features (duplicates subarrays and quadruplicates spots in each subarray) was averaged for each feature and sample, and the resulting cytokine values were determined. For every slide, a set of blanks was run as a negative control.

Data Quality Control: Subarray(s) were excluded from analysis if fluorescent intensities were generally weak (indicating weak RCA in that particular subarray), if there were visible defects in the array (such as scratches), or if there was high background signal. A total of 168 subarrays (84 samples) were analyzed, 4 subarray-1s and 4 subarray-2s were excluded on the basis of these quality control criteria. None of the samples failed both duplicated subarrays. Analyses were performed using complete set of data containing the levels of all 78 cytokines from 84 cell culture supernatants. Untransformed fluorescent intensities were used as data values in all of the analyses.

### Example 2

### Suppression by PARC

The results of Experiment 1, described in Example 1 above, measured at 48 hours are shown in FIGS. 2 and 3. These data demonstrate that the chemokine PARC suppresses expression of a set of cytokines including AR, IL-1sR1, IL-2, IL-6sR, LIF, AND SDF1a. Therefore PARC has utility in diseases characterized by too much mitosis, such as coronary artery disease, rheumatoid arthiritis, cancers, autoimmune diseases and myeloproliferative disorders.

### REFERENCES

1. Sorrentino, S., Human extracellular ribonucleases: multiplicity, molecular diversity and catalytic properties of the major RNase types. Cell Mol Life Sci, 1998. 54(8): p. 785-94.

2. Thomas, L. L. and S. M. Page, Inflammatory cell activation by eosinophil granule proteins. Chem Immunol, 2000. 76: p. 99-117.

3. Domachowske, J. B., et al., Eosinophil cationic protein/RNase 3 is another RNase A-family ribonuclease with direct antiviral activity. Nucleic Acids Res, 1998. 26(14): p. 3358-63.

4. Domachowske, J. B., et al., Evolution of antiviral activity in the ribonuclease A gene superfamily: evidencefor a specific interaction between eosinophil-derived neurotoxin (EDN/RNase 2) and respiratory syncytial virus. Nucleic Acids Res, 1998. 26(23): p. 5327-32.

5. Zheutlin, L. M., et al., Stimulation of basophil and rat mast cell histamine release by eosinophil granule-derived cationic proteins. J Immunol, 1984. 133(4): p. 2180-5.

6. Altman, L. C., et al., Cytokines and eosinophil-derived cationic proteins upregulate intercellular adhesion molecule-1 on human nasal epithelial cells. J Allergy Clin Immunol, 1993. 92(4): p. 527-36.

7. Minnicozzi, M., et al., Eosinophil granule proteins increase microvascular macromolecular transport in the hamster cheek pouch. J Immunol, 1994. 153(6): p. 2664-70.

8. Patella, V., et al., Eosinophil granule proteins activate human heart mast cells. J Immunol, 1996. 157(3): p. 1219-25.

9. Chihara, J., et al., Eosinophil cationic protein induces insulin-like growth factor I receptor expression on bronchial epithelial cells. Int Arch Allergy Immunol, 1996. 111 Suppl 1: p. 43-5.

10. Kimata, H., et al., Human B-cell growth-inhibitory activity of eosinophil cationic protein. Biotechnol Ther, 1992. 3(3-4): p. 137-49.

11. Kimata, H., et al., Eosinophil cationic protein inhibits immunoglobulin production and proliferation in vitro in human plasma cells. Cell Immunol, 1992. 141(2): p. 422-32.

12. El-Shazly, A. and T. Ishikawa, Novel co-operation between eotaxin and substance-P in inducing eosinophil-derived neurotoxin release. Mediators Inflamm, 1999. 8(3): p. 177-9.

13. El-Shazly, A., et al., Human eotaxin induces eosinophil-derived neurotoxin release from normal human eosinophils. Int Arch Allergy Immunol, 1998. 117 Suppl 1: p. 55-8.

14. Ohashi, H., et al., Effect of interleukin-3, interleukin 5 and hyaluronic acid on cultured eosinophils derivedfrom human umbilical cord blood mononuclear cells. Int Arch Allergy Immunol, 1999.118(1): p. 44-50.

15. Nakajima, H., et al., Production of IL-8 and release of eosinophil-derived neurotoxin by normalperipheral blood eosinophils. Int Arch Allergy Immunol, 1997. 114 Suppl 1: p. 36-9.

16. Kelly, E. A., W. W. Busse, and N. N. Jarjour, Inhaled budesonide decreases airway inflammatory response to allergen. Am J Respir Crit Care Med, 2000. 162(3 Pt 1): p. 883-90.

17. Tiffany, H. L., F. Li, and H. F. Rosenberg, Hyperglycosylation of eosinophil ribonucleases in a promyelocytic leukemia cell line and in differentiated peripheral bloodprogenitor cells. J Leukoc Biol, 1995. 58(1): p. 49-54.

18. Paul, C. C., et al., Cytokine induction of granule protein synthesis in an eosinophil-inducible human myeloid cell line, AML14. J Leukoc Biol, 1994. 56(1): p. 74-9.

19. Tsuda, S. and M. Miyasato, [Eosinophil granule proteins (MBP, ECP, EPX/EDN, EPO)—a possible process of eosinophil activation and degranulation]. Nippon Rinsho, 1993. 51(3): p. 613-9.

20. Fujisawa, T., et al., Regulatory effect of cytokines on eosinophil degranulation. J Immunol, 1990. 144(2): p. 642-6.

21. Schweitzer, B., et al., Multiplexed protein profiling on microarrays by rolling-circle amplification. Nat Biotechnol, 2002. 20(4): p. 359-65.

22. Naaby-Hansen, S., M. D. Waterfield, and R. Cramer, Proteomics—post-genomic cartography to understand genefunction. Trends Pharmacol Sci, 2001. 22(7): p. 376-84.

23. Figeys, D. and D. Pinto, Proteomics on a chip: promising developments. Electrophoresis, 2001. 22(2): p. 208-16.

24. Emili, A. Q. and G. Cagney, Large-scale functional analysis using peptide or protein arrays. Nat Biotechnol, 2000. 18(4): p. 393-7.

25. Hu, G.-F., Riordan, J. F., Vallee, B. L., A putative angiogenin receptor in angiogenin-responsive human endothelial cells. Proc. Nat. Acad. Sci. USA, 1997. 94: p. 2204-2209.

26. Hatzi, E., Badet, J., Expression of receptors for human angiogenin in vascular smooth muscle cells. Eur. J. Biochem., 1999.260: p. 825-832.

27. Gho, Y. S. Chae, C.-B., Anti-angiogenin activity of the peptides complementary to the receptor binding site of angiogenin. J. Biochem., 1997. 272(39): p. 24294-24299.

We claim:

1. A method of treating a disease characterized by an undesirable amount of cell proliferation, comprising:
administering an effective amount of chemokine PARC to a patient who has a proliferative cell disorder, whereby undesirable proliferation of cells is reduced.

2. The method of claim 1 wherein the syndrome is cancer.

3. The method of claim 1 wherein the syndrome is an autoimmune disease.

4. The method of claim 1 wherein the syndrome is rheumatoid arthritis.

5. The method of claim 1 wherein the syndrome is an myeloproliferative disease.

6. The method of claim 1 wherein the syndrome is coronary artery disease.

7. A method of diagnosing a proliferative cell disorder in a patient, comprising:
determining in a test sample from a patient amount of PARC; and
comparing the amount to an average amount found in a control sample of a population of healthy humans, wherein a decreased amount in the test sample relative to the average amount indicates an proliferative cell disorder in the patient.

8. The method of claim 7 wherein the syndrome is cancer.

9. The method of claim 7 wherein the syndrome is an autoimmune disease.

10. The method of claim 7 wherein the syndrome is rheumatoid arthritis.

11. The method of claim 7 wherein the syndrome is a myeloproliferative disease.

12. The method of claim 7 wherein the syndrome is coronary artery disease.

13. The method of claim 7 wherein the test sample is selected from the group consisting of serum, plasma, lymph fluid, peripheral lymphatic tissue, and blood.

14. The method of claim 7 wherein the test sample comprises dendritic cells.

15. The method of claim 7 wherein the test sample comprises Langerhans cells.

16. The method of claim 7 wherein the test sample comprises monocytes.

17. The method of claim 7 wherein an increased amount is at least two-fold more in the test sample than in the control sample.

18. The method of claim 7 wherein an increased amount is at least three-fold more in the test sample than in the control sample.

19. The method of claim 7 wherein an increased amount is at least four-fold more in the test sample than in the control sample.

20. The method of claim 7 wherein said step of determining employs an array of a first set of antibodies for capturing PARC.

21. The method of claim 20 wherein said step of determining employs a second set of antibodies which is applied to the array after binding of PARC in the test sample to the first set of antibodies.

22. The method of claim 21 wherein said second set of antibodies comprises covalently attached oligonucleotides.

23. The method of claim 21 wherein a third set of antibodies is applied to the array that specifically binds to the second set of antibodies.

24. The method of claim 23 wherein the third set of antibodies comprises covalently attached oligonucleotides.

25. The method of claim 24 wherein rolling circle amplification is performed using said oligonucleotides as primers.

26. A method of stimulating mitosis, comprising:
administering an inhibitory molecule for chemokine PARC to a subject in need of augmented mitosis, wherein the inhibitory molecule is selected from the group consisting of antibodies specific for chemokine PARC and antisense molecules which bind to and inhibit transcription of PARC mRNA, whereby mitosis in the subject is increased.

27. A method to monitor the effects of PARC or anti-PARC therapy, comprising:
(a) measuring amount of one or more protein selected from the group consisting of AR, IL-1sR1, IL-2, IL-6sR, LIF, SDF-1a, and fragments thereof in a sample collected from a patient at a first time;
(b) repeating step (a) in a sample collected from the patient at a later time;
(c) comparing the amounts measured in step (a) and in step (b), wherein an decreased amount over time denotes an effect of PARC and a increased amount denotes an effect of an anti-PARC inhibitory molecule.
